(19) United States
(12) Patent Application Publication
(10) Pub. No.: US 2022/0226426 A1
(43) Pub. Date: Jul. 21, 2022

(54) OPHTHALMIC COMPOSITION FOR THE TREATMENT OF OCULAR ALLERGY

(71) Applicant: NOVALIQ GMBH, Heidelberg (DE)

(72) Inventors: Frank LÖSCHER, Schriesheim (DE); Markus BEIER, Weinheim (DE); Chiara Silvana LEO, Heidelberg (DE)

(21) Appl. No.: 17/613,832

(22) PCT Filed: May 23, 2020
(86) PCT No.: PCT/EP2020/064346

§ 371 Date: Nov. 23, 2021

(30) Foreign Application Priority Data
May 24, 2019 (EP) 19176376.2

(51) Int. Cl. A61K 38/13; A61K 9/08; A61K 47/06; A61K 47/10; A61K 9/00; A61P 27/02

(52) U.S. Cl. CPC A61K 38/13; A61K 9/08; A61P 27/02; A61K 47/10; A61K 9/0048; A61K 47/06

(57) ABSTRACT

The invention provides ophthalmic composition comprising cyclosporine and a semifluorinated alkane for use in the treatment of ocular allergy and any symptoms associated thereto.

### BACKGROUND OF THE INVENTION

Ocular allergies encompass a group of hypersensitivity disorders to normally harmless substances, known as allergens and can be observed as the only dominant presentation of an allergic sensitisation, or are associated with rhinitis, asthma, atopic dermatitis or food allergy. The most common clinical presentations of ocular allergy are conjunctival hyperaemia (redness), chemosis (swelling), itching, tearing, and vision loss in severe cases.

The eye, particularly the conjunctiva, has a large number of mast cells. When allergens are present, they can bind to immunoglobulins on the surface of the mast cells and trigger their degranulation or breakdown. Many components, including histamines, are released through degranulation into the environment outside the mast cells. These components cause through a variety of mechanisms ocular surface inflammation, resulting in itching, lid edema, lid redness, tearing and photophobia. To alleviate these symptoms, histamine receptor antagonists or mast cell stabilisers are frequently used.

Seasonal allergic conjunctivitis (SAC) is the most common form of all ocular allergy disease, and is usually triggered by exposure to airborne pollens produced by plants that cause hay fever, the signs and symptoms typically occurring in spring and summer.

Perennial allergic conjunctivitis (PAC) is milder than SAC, and is a chronic condition that occurs throughout the year, being induced by exposure to dust, mites, fungi, animal epithelial and/or occupational allergens.

Vernal keratoconjunctivitis (VKC) is a self-limiting, chronic allergic inflammation of the ocular surface that typically affects young people and is usually more common in warm tropical climates.

Atopic keratoconjunctivitis (AKC) is a bilateral chronic inflammatory disease of the ocular surface and eyelids.

Contact dermatoconjunctivitis (CDC), contact allergy or allergic contact dermatitis is a type-IV hypersensitivity reaction, and occurs through interaction of an antigen with Th1 and Th2 cell subsets followed by a release of cytokines. Allergens are generally simple chemicals that combine with skin protein to form complete allergens, with examples including poison ivy, neomycin, latex, atropine and its derivatives. Contact allergy involves the ocular surface, eyelids and periocular skin, with the initial sensitization with a contact allergen taking several days. The reaction may peak 2-5 days after re-exposure, the delayed reaction being due to the slow migration of lymphocytes to the antigen depot. Withdrawing and avoiding contact with the allergen is effective in treating CDC, however, severe cases may require topical or systemic corticosteroids.

The diagnosis of ocular allergy is confirmed by a clinical history of typical eye symptoms, as well as in-vivo or in-vitro tests directed towards detecting free or cellbound IgE.

Several proteases, such as tryptase, tissue plasminogen activators and matrix metalloproteinase (MMP), have been found to be overexpressed in tears and tissues affected by VKC. Tryptase may be implicated in the activation of other proteases, such as MMPs, which are all involved in extracellular matrix degradation and inflammatory cell infiltration. MMP-9 (matrix metallopeptidase 9) activity correlates significantly with corneal involvement and giant papillae formation. Greater levels and activity of MMP correlated with clinical findings in patients with VKC, suggesting that proteases are involved in allergic inflammation (A. Leonardi, Experimental Eye Research 117, (2013), 106-117).

Treatment options for symptomatic ocular allergy include avoidance of the allergen, cold compressors, artificial tears, oral anti-allergies, vasoconstrictor/histamine eye drops, mast cell stabilisers eye drops, NSAIDS, corticosteroids and immunosupressives based on the severity of signs and symptoms.

BenEzra et al. American Journal of Ophthalmology 101:278-282, March 1986, describe a study aimed at evaluating the effect of 2% cyclosporine in olive oil eyedrops on the clinical course and symptoms of severe chronic vernal keratoconjunctivitis.

Cyclosporins have been used to treat inflammatory conditions. Cyclosporine is available, at least in the US, as an approved medicine in the form of an ophthalmic (o/w) emulsion (Restasis®). This product is indicated to increase tear production in patients whose tear production is presumed to be suppressed due to ocular inflammation associated with keratoconjunctivis sicca.

Ozcan et al. in Cornea, volume 26, Number 9, October 2007 describe a study aimed at evaluating the efficacy of topical cyclosporin A 0.05% in the treatment of vernal keratoconjunctivitis and atopic keratoconjunctivitis.

WO2011/073134 A1 describes pharmaceutical compositions in the form of solutions comprising cyclosporine and a semifluorinated alkane as a liquid vehicle which may be administered to the eye of a patient, such as for the treatment of keratoconjunctivitis sicca, for instance compositions comprising cyclosporine in semifluorinated alkane 1-(perfluorobutyl)pentane (F4H5) in the presence of ethanol as a co-solvent. WO2011/073134 A1 however does not describe treatment of ocular allergy.

It is thus an object of the present invention to provide ophthalmic compositions for use in the treatment of ocular allergy and associated conditions. Further objects of the invention will be clear on the basis of the following description of the invention, examples and claims.

### SUMMARY OF THE INVENTION

In a first aspect, the invention relates to an ophthalmic composition for use in the treatment of ocular allergy and any symptoms associated thereto, wherein the composition comprises cyclosporine and a semifluorinated alkane. In yet a further aspect, the invention provides for a kit comprising an ophthalmic composition for such uses.

### DESCRIPTION OF THE DRAWINGS

FIG. 1. Conjunctival lissamine green staining (change from baseline on Oxford scale). Depicted is the change from baseline of the conjunctival lissamine green staining value (mean) at 4 and 12 weeks of treatment (2 times per day) with vehicle (F4H5) and CyclAsol 0.1% w/v ophthalmic solution (ophthalmic solution of 1 mg/ml cyclosporine A dissolved in 1-(perfluorobutyl)pentane with 1.0% (w/w) ethanol) for the entire population of subjects. Error bars show the standard error of the mean (SEM).

FIG. 2. InflammaDry® MMP9 Test. Depicted is the percentage of subjects positive to the test at baseline and after 4 weeks of treatment (2 times per day) with vehicle (F4H5) and CyclAsol 0.1% w/v (ophthalmic solution of cyclosporine A dissolved in 1-(perfluorobutyl)pentane with 1.0% (w/w) ethanol).

### DETAILED DESCRIPTION OF THE INVENTION

The present invention relates to, in a first aspect, an ophthalmic composition for use in a method of treating ocular allergy and any symptoms or condition associated thereto, wherein the composition comprises cyclosporine and a semifluorinated alkane.

Eye allergy, also called allergic conjunctivitis, is quite common and occurs when the eyes react to an allergen. The eyelid and conjunctiva become swollen, red and itchy. In the present invention, ocular allergy may be one selected from seasonal allergic conjunctivitis, perennial allergic conjunctivitis, vernal keratoconjunctivitis, atopic keratoconjunctivitis and contact dermatoconjunctivitis. In a preferred embodiment, the ocular allergy is one selected from seasonal allergic conjunctivitis, perennial allergic conjunctivitis and vernal keratoconjunctivitis. In a more preferred embodiment, ocular allergy is one selected from seasonal allergic conjunctivitis and vernal keratoconjunctivitis.

Symptoms of ocular allergy are for example itching, lid edema, lid redness, burning, tearing and photophobia. In a preferred embodiment of the present invention, the symptoms associated with ocular allergy are selected from conjunctival hyperaemia (redness), chemosis (swelling), itching and tearing.

Cyclosporine (synonyms include cyclosporin A, CsA, or ciclosporin) is a cyclic nonribosomal peptide comprising 11 amino acids with the empirical formula C₆₂H₁₁₁N₁₁O₁₂ and molecular weight of 1202.61. It is an immunosuppressant drug that is widely used in post-allergenic organ transplant, to reduce the activity of the patient's immune system and thereby, the risk of organ rejection. Cyclosporine is typically provided as a colourless or white powder.

Cyclosporine is thought to bind to the cytosolic protein cyclophilin (immunophilin) of immunocompetent lymphocytes, especially T-lymphocytes. This complex of cyclosporin and cyclophilin inhibits calcineurin, which, under normal circumstances, is responsible for activating the transcription of interleukin 2. It also inhibits lymphokine production and interleukin release and, therefore, leads to a reduced function of effector T-cells.

In the present invention, the opthalmic composition comprises a semifluorinated alkane. The term “semifluorinated alkane” (also referred to as “SFA” throughout this document) as used herein refers to a linear or branched compound composed of at least one perfluorinated segment (F-segment) and at least one non-fluorinated hydrocarbon segment (H-segment). Preferably, the semifluorinated alkane is a linear or branched compound composed of one perfluorinated segment (F-segment) and one non-fluorinated hydrocarbon segment (H-segment). Preferably, said semifluorinated alkane is a compound that exists in a liquid state within the temperature range of 4° to 40° C. In one embodiment, the perfluorinated segment and/or the hydrocarbon segment of the said SFA optionally comprises or consists of a cyclic hydrocarbon segment, or optionally said SFA comprises an unsaturated moiety within the hydrocarbon segment.

It is preferred that the F- and the H-segment of the linear or branched semifluorinated alkane comprise, independently from one another, 2 to 10 carbon atoms.

According to a preferred embodiment of the present invention, the semifluorinated alkane is a linear compound of the formula (I) CF3(CF2)n(CH2)mCH3, wherein n and m are integers independently selected from each other from the range of 2 to 10, preferably selected from the range of 2 to 8 and even more preferably selected from the range of 3 to 7. More preferred is a semifluorinated alkane selected from the group consisting of F4H5, F4H6, F4H8, F6H6, F6H8 and F8H8.

Optionally, the linear or branched SFA may comprise a branched non-fluorinated hydrocarbon segment comprising one or more alkyl groups selected from the group consisting of —CH3, —C2H5, —C3H7 and —C4H9 and/or the linear or branched SFA may comprise a branched perfluorinated hydrocarbon segment, comprising one or more perfluorinated alkyl groups selected from the group consisting of —CF3, —C2F5, —C3F7 and —C4F9.

According to another nomenclature, the linear semifluorinated alkane may be referred to as FnHm, wherein F means the perfluorinated hydrocarbon segment, H means the non-fluorinated hydrocarbon segment and n, m is the number of carbon atoms of the respective segment. For example, F4H5 is used for 1-perfluorobutyl-pentane.

In a preferred embodiment of the present invention, the semifluorinated alkane is a semifluorinated alkane of formula (I) wherein n is selected from 3 to 5 and m is selected from 3 to 7. Preferably, the semifluorinated alkane is one selected from F4H5 and F6H8, more preferably F4H5.

The opthalmic composition for the use of the present invention may comprise from about 95 to about 99% wt.-%, more preferably from about 98 to about 99% wt.-%, of a semifluorinated alkane as described above, based on the total weight of the composition.

The opthalmic composition for use according to the present invention may comprise at least about 97% (w/w), preferably at least about 98% (w/w), more preferably at least about 99% (w/w) of a semifluorinated alkane, based on the total weight of the opthalmic composition.

Preferably, the opthalmic composition for the use of the present invention is formulated as a solution, more preferred as a clear solution.

The term a “clear solution”, as mentioned above and understood herein, refers to a liquid solution in which all solutes are fully dissolvable or dissolved under room temperature conditions i.e. between 15 and 25° C. The clear solution does not comprise of any particulate or solid phase components and preferably has a refractive index approximate to that of water (i.e. 1.333) at room temperature.

The concentration of cyclosporine in the opthalmic composition for use according to the invention may be in the range of from 0.05% (w/v) to about 0.25% (w/v), preferably in the range of from about 0.05% to 0.15% (w/v), more preferably in the range of from 0.05% to 0.10% (w/v) with respect to the total volume of the composition. In a preferred embodiment, the concentration of cyclosporine in the opthalmic composition for use according to the invention is one selected from 0.05% (w/v) and 0.10% (w/v), preferably 0.10% (w/v) with respect to the total volume of the composition.

Unless otherwise indicated, the term “% (w/v)” denotes the amount of a component of a composition as a weight percentage in relation to the total volume of the composition (with ‘w’ denoting the weight and ‘v’ denoting volume). For example 0.05% (w/v) may be understood as relating to 0.5 mg of a component in 1 mL of the composition, and 0.1% (w/v) would correspond to 1.0 mg of a component in 1 mL of the composition. Unless otherwise indicated, the term “% (w/w)” refers to the amount of a component of a composition as a weight percentage in relation to the total weight of the composition (with ‘w’ denoting weight).

The term ‘about’ as used herein and in reference or connection to a parameter, for example such as the concentration of cyclosporine dissolved in the composition or the amount of cyclosporine featured in a single dose of the composition includes the precise value as defined, as well as any value falling within the degree of variability usually observed in measuring or determining these parameters using the standard techniques and equipment known in the art and field.

A single dose of the opthalmic composition for the use of the present invention may be administered in a volume of about 8-12 μl, preferably in a volume of about 10-12 μl, more preferably 11-12 μl, most preferably about 11 μl.

A dose of a composition for use according to the present invention and as described in any one of the embodiments herein is preferably topically administered in the form of a (i.e. one) single drop to an eye of a subject. The drop may be administered to the surface of the eye, preferably to any surface region or tissue of the eye that is accessible to topical administration or instillation, for example to the cornea or conjunctiva. The single drop of the composition may be instilled directly onto a surface of the eye, such as the corneal surface of the eye, or alternatively into a space i.e. sac or pocket formed by gently pulling down of the lower eyelid of an eye.

As used herein, the term ‘administration to an eye’ or ‘per eye’ refers to the administration of a given dose, e.g. a single dose, of a opthalmic composition according to the invention to an individual eye of a subject. The therapy of ocular allergy and symptoms or associated conditions as described herein however, should be understood as being not limited to the treatment of a single eye in a subject, but as being also inclusive of a therapy involving the administration of composition according to the present invention to each i.e. both eyes of a subject which are affected by ocular allergy.

Preferably, the ophthalmic composition for use according to the present disclosure is topically administered between one to four times per day, more preferably between one and two times per day, even more preferably two times per day to the eye of a subject.

In the present invention, the opthalmic composition may also comprise one or more further excipients as an optional and additional component. The term “excipients” as used herein refers to any pharmaceutically acceptable natural or synthetic substance that may be added to the ophthalmic composition to enhance or otherwise modify its physical or chemical constitution or stability or therapeutic properties. The present opthalmic composition may optionally comprise one or more excipients such as, for example, an antioxidant, a preservative, a lipid or oily excipient, a surfactant or a lubricant or a combination of at least 2 excipients thereof.

Suitable antioxidants for use in the present opthalmic composition comprise, for example: butylated hydroxytoluene (BHT), butylated hydroxyanisole (BHA), tertiary butylhydroquinone (TBHQ), vitamin E, vitamin E derivatives (i.e. alpha-tocopherol acetate) or ascorbic acid.

Suitable lipid or oily excipients for use in the present opthalmic composition comprise, for example, triglyceride oils (i.e. soybean oil, olive oil, sesame oil, cotton seed oil, castor oil, sweet almond oil), triglycerides, mineral oil (i.e. petrolatum and liquid paraffin), medium chain triglycerides (MCT), oily fatty acids, isopropyl myristate, oily fatty alcohols, esters of sorbitol and fatty acids, oily sucrose esters, or any other oily substance which is physiologically tolerated by the eye.

Suitable lubricants for use in the present opthalmic composition comprise, for example, carboxymethylcellulose and its sodium salt (CMC, carmellose), polyvinyl alcohol, hydroxypropyl methylcellulose (HPMC, hypromellose), hyaluronic acid and its sodium salt, or hydroxypropyl guar gum.

The opthalmic composition according to the present invention may or may not comprise pharmaceutically suitable natural or synthetic preservatives, such as, for example, benzalkonium chloride and chlorhexidine. In a preferred embodiment, however, the opthalmic composition according to the present invention does not comprise a pharmaceutically acceptable preservative.

In addition to the excipients as described above as optional components, the present opthalmic composition may also comprise one or more further solvents.

The term “further solvents” as used herein refers to a solvent or mixture of two or more different solvents other than the semifluorinated alkane. Suitable further solvents may be chosen from, for example, alcohols, such as ethanol, isopropanol or other further solvent which is physiologically tolerated by the eye, such as transcutol.

The opthalmic composition for the use of the present invention may comprise a further solvent selected from ethanol or transcutol, preferably ethanol. In a preferred embodiment, the opthalmic composition for the use of the present invention comprises a further solvent in an amount of up to 1.5% (w/w) with respect to the total weight of the composition. In a more preferred embodiment, the opthalmic composition for the use of the present invention comprises ethanol or transcutol in an amount of up to 1.wt %.

Ethanol may be present in the opthalmic composition for use according to the present invention in an amount of up to about 1.5 wt.-%, preferably up to about 1.0 wt.-%, such as, for example from 0.2 to 1.0 wt.-% (corresponding to 0.2% to 1.0% (w/w)) or 0.5 to 1.0 wt.-% (corresponding to 0.5 to 1.0% (w/w)), based on the total weight of the composition (final dosage form). Preferably, the opthalmic composition for use according to the present invention comprises about 0.5 to 1.0 wt.-% ethanol, more preferably about 1.0 wt.-% ethanol with respect to the total weight of the opthalmic composition.

In a preferred embodiment, the opthalmic composition for the use of the present invention is essentially free of water, whereas the residual water may be attributed to the potential residual water content of cyclosporin. The term ‘essentially’ as used herein means if present then in trace or residual amounts such as to confer no technical advantage or relevance in respect of the object of the invention.

As used herein, the term “up to about” or “up to” used in context of a parameter, refers to any value of the parameter greater than zero and up to, and inclusive of, the defined parameter. For example, an amount of “up to about 1.0% (w/w) of ethanol” should be understood as including any value greater than zero ranging up to and including the value of 1.0% (w/w) of ethanol, and would include, for example, values such as 0.01%, 0.05%, 0.1%, 0.2%, 0.3%, 0.4%, 0.5% 0.6%, 0.7, 0.8, 0.9, 0.95%, 0.99% (w/w) of ethanol, taking into account any degree of variability usually observed in measuring or determining this parameter, using the standard techniques and equipment known in the relevant field.

In another preferred embodiment, the opthalmic composition for the use of the present invention is (essentially) water-free and/or preservative free.

In one embodiment of the invention, the composition for the use according to the invention may comprise about 0.05% to 0.1% (w/v) cyclosporine dissolved in 1-(perfluorobutyl)pentane, and about 1.0% (w/w) of ethanol based on the total weight of the composition.

In another preferred embodiment, the opthalmic composition for the use of the present invention consists essentially of about 0.05% to 0.1% (w/v) cyclosporine dissolved in 1-(perfluorobutyl)pentane and about 1.0% (w/w) ethanol based on the total weight of the composition.

In preferred embodiments of the invention, the composition for use as described herein may preferably comprise, or consist of:

0.05 to 0.1% (w/v) of cyclosporine dissolved in 1-(perfluorobutyl)pentane and 0.5% (w/w) ethanol, or 0.05 to 0.1% (w/v) of cyclosporine dissolved in 1-(perfluorobutyl)pentane and 1.0% (w/w) ethanol, or 0.05% (w/v) cyclosporine dissolved in 1-(perfluorobutyl)pentane and 0.5% (w/w) ethanol, or 0.1% (w/v) cyclosporine dissolved in 1-(perfluorobutyl)pentane and 0.5% (w/w) ethanol, or 0.1% (w/v) cyclosporine dissolved in 1-(perfluorobutyl)pentane and 1.0% (w/w) ethanol, or 0.05% (w/v) cyclosporine dissolved in 1-(perfluorobutyl)pentane and 1.0% (w/w) ethanol, or 0.05 to 0.1% (w/v) of cyclosporine dissolved in 1-(perfluorobutyl)pentane, or 0.1% (w/v) cyclosporine dissolved in 1-(perfluorobutyl)pentane, or 0.05% (w/v) cyclosporine dissolved in 1-(perfluorobutyl)pentane, or 0.05 to 0.25% (w/v) cyclosporine dissolved in 1-(perfluorobutyl)pentane and 1.0% (w/w) ethanol, or 0.05 to 0.25% (w/v) cyclosporine dissolved in 1-(perfluorobutyl)pentane, or 0.1 to 0.25% (w/v) cyclosporine dissolved in 1-(perfluorobutyl)pentane and 1.0% (w/w) ethanol, or 0.1 to 0.25% (w/v) cyclosporine dissolved in 1-(perfluorobutyl)pentane, or 0.05 to 0.25% (w/v) cyclosporine dissolved in 1-(perfluorobutyl)pentane and 0.5% (w/w) ethanol, or 0.1 to 0.25% (w/v) cyclosporine dissolved in 1-(perfluorobutyl)pentane and 0.5% (w/w) ethanol.

In a preferred embodiment, the opthalmic composition for the use of the present invention is a clear solution comprising cyclosporin at a concentration of from about 0.05% (w/v) to 0.25% (w/v), preferably from about 0.05% (w/v) to 0.1% (w/v) dissolved in 1-(perfluorobutyl)pentane and up to about 1% (w/w) ethanol (at room temperature conditions i.e. between 15 to 25° C.). In a preferred embodiment, the opthalmic composition for the use of the present invention is provided in sterile form.

Preferably, the opthalmic composition for use according to the present invention are substantially free of water, substantially free of a preservative and are effective in inhibiting microbal growth.

Preferably, the opthalmic composition for use according to the present invention form small droplets (drops), in the range of about 8-12 μl, more preferably about 10-12 μl, even more preferably about 11-12 μl, most preferably about 11 μl, when administered from a drop dispenser.

As used herein, the term “consists” and related terms “consisting” or “consist” is to be understood as meaning that no other features, other than those prefaced by the term are present. In the context of opthalmic compositions, if any other constituent or component is present in the composition other than those prefaced by such term, then it is present only in trace or residual amounts such as to confer no technical advantage or relevance in respect of the object of the invention, such as may be further understood by the term ‘essentially” or “substantially” used in conjunction with these terms (e.g. ‘essentially consisting of”).

In a further embodiment, the composition for use in a method of treating ocular allergy and any symptoms associated thereto, comprises 0.05% to 0.10% (w/v) cyclosporine dissolved in 1-(perfluorobutyl)pentane, and optionally up to about 1 wt % ethanol, and is administered between one and four times per day at a single dose of about 8-12 μl, preferably at a single dose of about 10-12 μl, per eye to a subject.

In a further embodiment, the composition for use in a method of treating ocular allergy and any symptoms associated thereto, comprises 0.05% (w/v) cyclosporine dissolved in 1-(perfluorobutyl)pentane, and optionally up to about 1 wt % ethanol, and is administered between one and four times per day at a single dose of about 8-12 μl, preferably at a single dose of about 10-12 μl, per eye to a subject.

In a further embodiment, the composition for use in a method of treating ocular allergy and any symptoms associated thereto, comprises 0.1% (w/v) cyclosporine dissolved in 1-(perfluorobutyl)pentane, and optionally up to about 1 wt % ethanol, and is administered between one and four times per day at a single dose of about 8-12 μl, preferably at a single dose of about 10-12 μl, per eye to a subject.

In a further embodiment, the composition for use in a method of treating ocular allergy and any symptoms associated thereto, comprises 0.05% to 0.10% (w/v) cyclosporine dissolved in 1-(perfluorobutyl)pentane, and optionally up to about 1 wt % ethanol, and is administered up to four times per day at a daily dose of between 20 to 48 μg, per eye to a subject.

In a further embodiment, the composition for use in a method of treating ocular allergy and any symptoms associated thereto, comprises 0.05% (w/v) cyclosporine dissolved in 1-(perfluorobutyl)pentane, and optionally up to about 1 wt % ethanol, and is administered up to four times per day at a daily dose of between to 24 μg, per eye to a subject.

In a further embodiment, the composition for use in a method of treating ocular allergy and any symptoms associated thereto, comprises 0.10% (w/v) cyclosporine dissolved in 1-(perfluorobutyl)pentane, and optionally up to about 1 wt % ethanol, and is administered up to four times per day at a daily dose of between to 48 μg, per eye to a subject.

In a further embodiment, the composition for use in a method of treating ocular allergy and any symptoms associated thereto, comprises 0.05% (w/v) cyclosporine dissolved in 1-(perfluorobutyl)pentane, and optionally up to about 1 wt % ethanol, and is administered two times per day at a daily dose of between 10 to 12 μg, per eye to a subject.

In a further embodiment, the composition for use in a method of treating ocular allergy and any symptoms associated thereto, comprises 0.10% (w/v) cyclosporine dissolved in 1-(perfluorobutyl)pentane, and optionally up to about 1 wt % ethanol, and is administered two times per day at a daily dose of between 20 to 24 μg, per eye to a subject.

In a further embodiment, the composition for use in a method of treating ocular allergy and any symptoms associated thereto, comprises 0.05% to 0.10% (w/v) cyclosporine dissolved in 1-(perfluorobutyl)pentane, and optionally up to about 1 wt % ethanol, and is administered at a daily dose of between 5.5 to 11 μg per eye when administered one time per day, or at a daily dose of between 11 to 22 μg per eye when administered two times per day; or at a daily dose of between 16.5 to 33 μg when administered three times per day; or at a daily dose of between 22 and 44 μg per eye when administered four times per day.

In a further embodiment, the composition for use in a method of treating ocular allergy and any symptoms associated thereto, comprises 0.05% (w/v) cyclosporine dissolved in 1-(perfluorobutyl)pentane, and optionally up to about 1 wt % ethanol, and is administered at a daily dose of between 5 to 6 μg per eye when administered one time per day, or at a daily dose of between 10 to 12 μg per eye when administered two times per day; or at a daily dose of between 15 to 18 μg per eye when administered three times per day; or at a daily dose of between 20 and 24 μg per eye when administered four times per day.

In a further embodiment, the composition for use in a method of treating ocular allergy and any symptoms associated thereto, comprises 0.1% (w/v) cyclosporine dissolved in 1-(perfluorobutyl)pentane, and optionally up to about 1 wt % ethanol, and is administered at a daily dose of between 10 to 12 μg per eye when administered one time per day, or at a daily dose of between 20 to 24 μg per eye when administered two times per day; or at a daily dose of between 30 to 36 μg per eye when administered three times per day; or at a daily dose of between 40 and 48 μg per eye when administered four times per day.

In a further embodiment, the composition for use in a method of treating ocular allergy and any symptoms associated thereto, comprises 0.05% to 0.10% (w/v) cyclosporine dissolved in 1-(perfluorobutyl)pentane, and optionally up to about 1 wt % ethanol, and is administered at a daily dose of between 5 to 12 μg per eye when administered one time per day, or at a daily dose of between 10 to 24 μg per eye when administered two times per day; or at a daily dose of between 15 to 36 μg per eye when administered three times per day; or at a daily dose of between 20 and 48 μg per eye when administered four times per day.

According to a further embodiment, the composition for use in a method of treating ocular allergy and any symptoms associated thereto is administered to a subject characterized by one or more symptoms selected from red eyes, irritated eyes, itching, burning sensation, sensitivity to light, swollen eyelids.

Preferably, the composition for use in a method of treating ocular allergy and any symptoms associated thereto is administered to a subject suffering from a comorbidity selected from dry eye disease, meibomian gland dysfunction, stye, chalazion, blepharitis, ectropion, eyelid laxity, eyelid edema, eyelid dermatitis, punctate keratopathy, nasal allergies, allergic skin condition or any combination thereo. More preferably the comorbidity the subject is suffering of is dry eye disease, preferably the comorbidity is selected from aqueous dry eye disease, meibomian gland disease associated with dry eye disease, evaporative dry eye disease, or a combination thereof.

Preferably, the composition for use in a method of treating ocular allergy and any symptoms associated thereto is effective to reduce the (ocular allergy associated) inflammation of the conjunctiva, preferably as determined by conjunctival lissamine green stating and/or assessing levels of MMP-9 (matrix metallopeptidase 9).

The use of an opthalmic composition as described in any one of the above embodiments in the manufacture or preparation of a medicament or a medicine for the treatment of a subject in need thereof in relation to any one of preferred ocular allergy conditions described herein are also provided for in the context of the present invention.

All the preferred embodiments described above in relation to the opthalmic composition for the use of the present invention apply to the use of the opthalmic composition for the manufacture or preparation of a medicament or a medicine for the treatment of a subject suffering from ocular allergy.

Further provided for within the context of the present invention, are also methods of treating subjects diagnosed with, and/or suffering from said ocular allergy as described herein, wherein the methods may comprise the topical administration, such as by direct topical instillation to the eye, of any one of the defined compositions.

All the preferred embodiments described above in relation to the opthalmic composition for the use of the present invention apply to the method of treating subjects diagnosed with and/or suffering from ocular allergy and any symptoms associated thereto.

Said treatment methods and compositions for therapeutic use are moreover preferably targeted towards human subjects diagnosed and/or suffering from ocular allergy.

In yet a further aspect, the invention provides also a kit comprising a opthalmic composition for use according to the invention and any of the embodiments described above, wherein the kit comprises a container for holding the opthalmic composition and a drop dispenser.

As understood herein, the drop dispenser may be a dispenser or applicator means which may be mounted, fixed or connected to the container for holding the opthalmic composition. Preferably, the drop dispenser is adapted for dispensing a single dose in the form of a single drop of the composition. More preferably, the drop dispenser is adapted for dispensing a single dose of 8- to 12-μl volume, preferably 10 to 12 μl, even more preferably 11 to 12 μl, most preferably a single dose of about 11-μl volume.

The container for holding the opthalmic composition as understood herein is preferably of a volume which may hold a single dose, but more preferably of a volume which may hold multiple or a plurality of doses of the composition. In an embodiment of the invention, the container of the kit may hold up to 250 doses of the opthalmic composition for use according to the present invention.

The container and/or the drop dispenser preferably may be manufactured from a thermoplastic material or polymer. In a one embodiment, the container and/or drop dispenser is manufactured from a thermoplastic material selected from polyethylene and polypropylene.

In one particular embodiment, the drop dispenser is manufactured from a polyethylene material, preferably selected from low density polyethylene and high density polyethylene, and more preferably is manufactured from a high-density polyethylene. In another embodiment, the container is manufactured from a polypropylene or polyethylene material, and more preferably is manufactured from polypropylene.

Particularly preferred are kits comprising a opthalmic composition for use in accordance with the present invention, wherein the kit comprises, in addition to a drop dispenser adapted for administering about 8 to 12 μl per drop, any one of the following:

about 2.0 ml of the opthalmic composition filled in a 3.0 ml volume container (i.e. a respective ratio of about 0.7); or about 2.0 ml of a opthalmic compostions filled in a 5.0 ml volume container (i.e. a respective ratio of about 0.4); or about 2.5 ml of a opthalmic compostions filled in a 5.0 ml volume container (i.e. a respective ratio of about 0.5).

Also preferred is a kit comprising a opthalmic composition for use in accordance with the present invention, wherein the kit comprises a container for holding the opthalmic composition and a drop dispenser adapted for administering about 8 to 12 μl per drop and wherein the ratio of the volume of head space in the container to the volume of the opthalmic composition is between 0.5 to 1.5. As understood herein, the volume of head space (or head space volume) in the container refers to the interior volume of the container, formed by the interior dimensions of the container which is not filled or occupied by the liquid opthalmic composition but which may contain atmosphere or inert gas.

For example, in a kit comprising a container holding a fill volume of 2.5 ml of a opthalmic composition for use according to the present invention, it is preferred that the head space volume available in the container is about 2.5 ml, wherein the ratio of the head space to opthalmic composition fill volume is about 1.0.

Particularly preferred are kits comprising a opthalmic composition for use in accordance with the present invention, wherein the kit comprises, in addition to a drop dispenser adapted for administering about 8 to 12 μl per drop, preferably about 10-12 μl per drop, more preferably 11-12 μl, most preferably 11 μl, any one of the following:

a container holding about 2.0 ml of the opthalmic composition, wherein the container has about 1.0 ml volume of head space (i.e. a head space to fill volume ratio of about 0.5); or a container holding about 2.0 ml of the opthalmic composition, wherein the container has about 3.0 ml volume of head space (i.e. a head space to fill volume ratio of about 1.5); or a container holding about 2.4 ml of the opthalmic composition, wherein the container has about 2.6 ml volume of head space (i.e. a head space to fill volume ratio of about 1.1).

Such kits as provided in accordance with these embodiments may improve storage and dispensability (i.e., ease and consistency in dispensing) of the opthalmic compositions.

Further, the present invention comprises the following items 1 to 10, relating to a method for treating ocular allergy:
1. A method of treating ocular allergy and any symptoms associated thereto, wherein the method comprises topically administering to an eye of a human suffering from ocular allergy a composition comprising cyclosporine and a semifluorinated alkane, wherein said method is therapeutically effective in treating ocular allergy and any symptoms related thereto in said human.
2. A method of treating ocular allergy according to Item 1, wherein ocular allergy is one selected from seasonal allergic conjunctivitis, perennial conjunctivitis, vernal keratoconjunctivitis, atopic keratoconjunctivitis and contact dermatoconjunctivitis.
3. A method of treating ocular allergy according to any preceding items, wherein cyclosporine is present at a concentration of from about 0.05% (w/v) to 0.1% (w/v), preferably of about 0.1% (w/v).
4. A method of treating ocular allergy according to any of the preceding items, wherein the composition comprises a further solvent, preferably ethanol as a further solvent.
5. A method of treating ocular allergy according to item 4, wherein ethanol is present at a concentration of up to about 1.0% (w/w) based on the total weight of the composition.
6. A method of treating ocular allergy according to any of the preceding items, wherein said composition consists of 0.05% to 0.1% (w/v) cyclosporine dissolved in a solution of 1-(perfluorobutyl)pentane and about 1.0% (w/w) ethanol.
7. A method of treating ocular allergy according to any preceding items, wherein said composition consists of 0.1% (w/v) cyclosporine dissolved in a solution of about 99% (w/w) 1-(perfluorobutyl)pentane and about 1% (w/w) ethanol.
8. A method of treating ocular allergy according to any preceding items, wherein said composition comprises up to about 0.5% (w/w) ethanol based on the total weight of the composition.
9. A method of treating ocular allergy according to item 8, wherein said composition consists of 0.05% to 0.1% (w/v) cyclosporine dissolved in a solution of about 99.5% (w/w) 1-(perfluorobutyl)pentane and about 0.5% (w/w) ethanol.
10. A method of treating ocular allergy according to item 8, wherein said composition consists of 0.1% (w/v) cyclosporine dissolved in a solution of at least about 99.5% (w/w) 1-(perfluorobutyl)pentane and up to about 0.5% (w/w) ethanol.
11. A method of treating ocular allergy according to any preceding items, wherein the symptoms associated with ocular allergy are selected from conjunctival hyperaemia (redness), chemosis (swelling), itching and tearing.

Further, the present invention comprises the following items 1 to 15, relating to a composition for use in a method for treating ocular allergy and any symptoms associated thereto:
1. An ophthalmic composition for use in a method of treating ocular allergy and any symptoms associated thereto, wherein the composition comprises cyclosporine and a semifluorinated alkane.
2. The composition for use according to item 1, wherein the ocular allergy is one selected from seasonal allergic conjunctivitis, perennial allergic conjunctivitis, vernal keratoconjunctivitis, atopic keratoconjunctivitis and contact dermatoconjunctivitis.
3. The composition for use according to any preceding items, wherein the symptoms associated with ocular allergy are selected from conjunctival hyperaemia (redness), chemosis (swelling), itching and tearing.
4. The composition for use according to any preceding items, wherein the semifluorinated alkane is one selected from 1-(perfluorobutyl)pentane and 1-(perfluorohexyl) octane.
5. The composition for use according to any preceding item, wherein the semifluorinated alkane is 1-(perfluorobutyl)pentane.
6. The composition for use according to any preceding item, wherein the composition comprises cyclosporine at a concentration of up to about 2.5 mg/ml.
7. The composition for use according to any preceding item, wherein the composition comprises cyclosporine at a concentration of about 0.5 mg/ml to about 1 mg/ml.
8. The composition for use according to any preceding item, further comprising a further solvent.
9. The composition for use of item 8, wherein the further solvent is one selected from ethanol and transcutol, preferably ethanol.
10. The composition for use according to item 9, wherein the further solvent is present at a concentration of up to 1.0% (w/w) with respect to the total weight of the composition.
11. The composition for use according to any preceding item, wherein the composition comprises cyclosporine at a concentration of 1.0 mg/ml dissolved in 1-(perfluorobutyl)pentane (F4H5).
12. The composition for use according to any preceding item, wherein the composition consists of about 0.5 mg/ml or 1.0 mg/ml cyclosporine dissolved in 1-(perfluorobutyl)pentane and up to about 1% (w/w) ethanol.
13. The composition for use according to item 12, wherein the composition consists of 1.0 mg/ml cyclosporine dissolved in 1-(perfluorobutyl)pentane and up to about 1% (w/w) ethanol.
14. The composition for use according to any preceding items, wherein the composition is formulated as a clear solution, preferably water-free and/or preservative free.
15. A kit comprising the ophthalmic composition for the use of any of the preceding items.

### EXAMPLES

The two following treatments 1) CyclASol 0.1% Ophthalmic Solution (Cyclosporine A dissolved in 1-(perfluorobutyl)pentane and ethanol 1.0% (w/w)) and 2) Vehicle Ophthalmic Solution (F4H5) were administered to patients participating to a study listed in clinicaltrials.gov under the number NCT03292809. Patients included in the study had to fulfil inter alia the following criteria, i.e. have a total lissamine green conjunctival score (sum of temporal and nasal) of 2, based on the Oxford grading at Visits 0 (Day −14±2 days, Screening) and Visit 1 (Day 1, Baseline/Randomization).

Subjects eligible to be randomized received one of the two treatments to dose with bilaterally BID for approximately 82 days from Visit 1 to Visit 5 (Day 85±2 days, 12-Week Follow-Up and Study Exit).

The full analysis set of patients were, respectively, 162 for CyclAsol 0.1% treatment and 166 for vehicle treatment. At baseline, the CyclAsol 01.% group of patients had a mean conjunctival staining of 4.1 (1.70). At baseline, the vehicle group of patients had a mean conjunctival staining of 4.3 (1.66).

Subjects were instructed to instill one drop of treatment 1) or 2) in each lower eyelid two times daily (in the morning and in the evening before bed).

Conjunctival Lissamine Green Staining

Conjunctival Lissamine Green Staining (Bron A. J. et al, Cornea. 2003; 22:640-650) was conducted by instillation of 10 μl of lissamine green solution into the inferior conjunctival cul-de-sac of a subject. After waiting for approximately 30 seconds the staining was evaluated. The subject was instructed to blink several times to distribute the lissamine green. The staining was graded with the Oxford Grading Scale. Herein, the lissamine staining is represented by punctate dots on a series of panels (A-E). Staining ranges from 0-5 for each panel and 0-10 for the total exposed inter-palpebral conjunctiva. Both nasal and temporal regions were graded separately. A score of 0 means no staining. Total conjunctival lissamine green staining scores were obtained, referring to the sum of scores from both temporal and nasal regions of the conjunctiva.

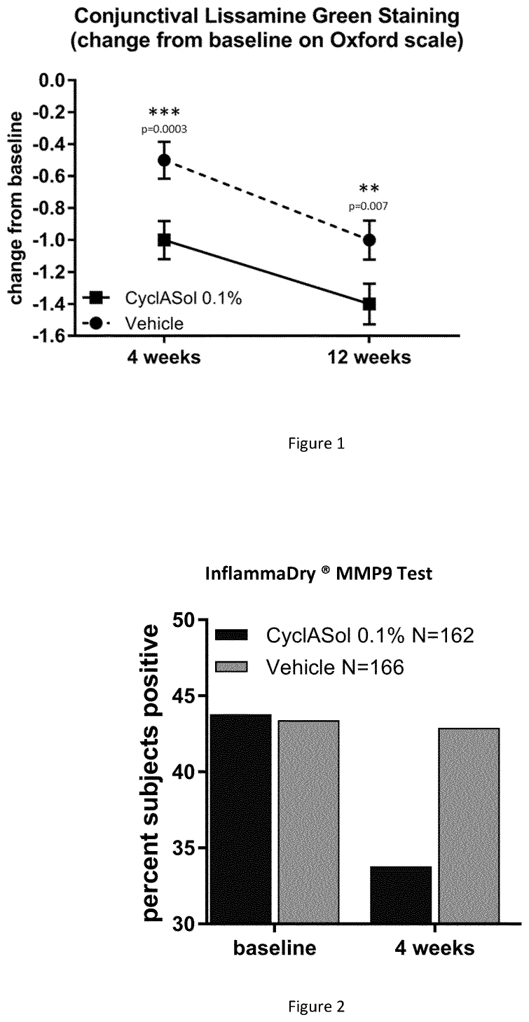

It was observed that subjects undergoing treatment with CyclAsol 0.1% w/v show a decrease of the conjunctival staining, indicating that the conjunctiva benefits of the CyclAsol treatment and ocular surface health can be improved (FIG. 1).

InflammaDry® MMP9 Test

The levels of MMP-9 were measured in each eye using InflammaDry® test. The test was recorded as either positive or negative. A sampling fleece was dabbed along the palpebral conjunctiva until saturated. Then, the sampling fleece was inserted in the test cassette to read the result, according to the general instructions of InflammaDry® test.

As shown in FIG. 2, after four weeks of treatment the levels of MMP-9 decreased significantly in patients who were positive to the test at baseline and underwent CyclAsol 0.1% w/v treatment, indicating that inflammation of the conjunctiva was reduced.

1. An ophthalmic composition for use in a method of treating ocular allergy and any symptoms associated thereto, wherein the composition comprises cyclosporine at a concentration of about 0.5 mg/ml to about 1 mg/ml dissolved in 1-(perfluorobutyl)pentane.

2. The composition for use according to claim 1, wherein the ocular allergy is one selected from seasonal allergic conjunctivitis, perennial allergic conjunctivitis, vernal keratoconjunctivitis, atopic keratoconjunctivitis and contact dermatoconjunctivitis.

3. The composition for use according to any preceding claims, wherein the symptoms associated with ocular allergy are selected from conjunctival hyperaemia (redness), chemosis (swelling), itching and tearing.

4. The composition for use according to any preceding claim, further comprising a further solvent selected from ethanol and transcutol.

5. The composition for use according to claim 4, wherein the further solvent is present at a concentration of up to 1.0% (w/w) with respect to the total weight of the composition.

6. The composition for use according to any preceding claim, wherein the composition comprises cyclosporine at a concentration of 0.5 mg/ml to 1.0 mg/ml dissolved in 1-(perfluorobutyl)pentane (F4H5), and optionally up to about 1.0% (w/w) ethanol.

7. The composition for use according to any preceding claim, wherein the composition consists of about 0.5 mg/ml or 1.0 mg/ml cyclosporine dissolved in 1-(perfluorobutyl)pentane and up to about 1% (w/w) ethanol.

8. The composition for use according to claim 7, wherein the composition consists of 1.0 mg/ml cyclosporine dissolved in 1-(perfluorobutyl)pentane and up to about 1% (w/w) ethanol.

9. The composition for use according to any preceding claim, wherein the composition is formulated as a clear solution, preferably water-free and/or preservative free.

10. The composition for use according to any preceding claim, wherein the composition is administered at a single dose of about 10-12 μl per eye.

11. The composition for use according to any preceding claims, wherein the composition is topically administered between one and four times per day to the eye of a subject.

12. The composition for use according to any preceding claim, wherein the composition is administered at a daily dose of
i.) between 5 to 12 μg per eye when administered one time per day
ii.) between 10 to 24 μg per eye when administered two times per day
iii.) between 15 to 36 μg per eye when administered three times per day; or
iv.) between 20 and 48 μg per eye when administered four times per day

13. The composition for use according to any preceding claim, wherein the subject is characterized by one or more symptoms selected from red eyes, irritated eyes, itching, burning sensation, sensitivity to light and swollen eyelids.

14. The composition for use according to any preceding claim, wherein the subject suffers from a comorbidity selected from dry eye disease, stye, chalazion, blepharitis, ectropion, eyelid laxity, eyelid edema, eyelid dermatitis, punctate keratopathy, nasal allergies, allergic skin condition or any combination thereof.

15. The composition for use according to claim 14, wherein comorbidity is dry eye disease, selected from aqueous dry eye disease, meibomian gland disease associated with dry eye disease, evaporative dry eye disease, or a combination thereof.

16. The composition for use according to any preceding claims, wherein the method is effective in reducing the inflammation of the conjunctiva.

17. A kit comprising the ophthalmic composition for the use of any of the preceding claims.
